(19) United States
(12) Plant Patent Application Publication
(10) Pub. No.: US 2018/0098474 P1
(43) Pub. Date: Apr. 5, 2018

(54) Hop plant named 'Furano K906901060 Go'

(50) ;

(71) Applicant: Sapporo Breweries Limited, Tokyo (JP)

(72) Inventors: Koichiro Koie, Hokkaido (JP); Narushi Suda, Hokkaido (JP); Yutaka Itoga, Hokkaido (JP); Mitsuhiro Uemoto, Hokkaido (JP); Yasunori Arai, Hokkaido (JP); Masanobu Goto, Hokkaido (JP)

(73) Assignee: Sapporo Breweries Limited, Tokyo (JP)

(21) Appl. No.: 15/731,660

(22) Filed: Jul. 13, 2017

(30) Foreign Application Priority Data
Oct. 5, 2016 (JP) 31508

(51) Int. Cl. A01H 5/02

(52) U.S. Cl. PLT/236

(57) ABSTRACT

A new hop plant particularly distinguished by having a cylindrical plant shape, large bracts, and a fruity, tropical fruit flavor, is disclosed.

### CROSS REFERENCE TO RELATED APPLICATION

The present application claims priority to Japan Plant Variety Protection Application No. 31508, as filed on Oct. 5, 2016, the entire contents are herein incorporated by reference for all the application teaches and discloses.

Genus and species: Humulus lupulus L.

Variety denomination: ‘Furano K906901060 Go’.

### BACKGROUND OF THE NEW PLANT

The present invention comprises a new and distinct variety of hop, botanically known as Humulus lupulus L. and hereinafter referred to by the variety name ‘Furano K906901060 Go’. ‘Furano K906901060 Go’ is a selection from a controlled cross-pollination conducted in Kamifurano, Hokkaido, Japan between the proprietary female hop variety ‘K843502179’ and the male hop variety ‘Saaz’ in 1990.

The seeds from the cross-pollination were sown in 1991 and plants were grown for evaluation. A plant line was selected in March, 2014 in Kamifurano, Hokkaido, Japan and named ‘Furano K906901060 Go’. In 2014, ‘Furano K906901060 Go’ was first vegetatively propagated in Kamifurano, Hokkaido, Japan via vegetative cuttings. ‘Furano K906901060 Go’ was found to reproduce true to type in successive generations of asexual propagation via vegetative cuttings in Kamifurano, Hokkaido, Japan.

### SUMMARY

The following are the most outstanding and distinguishing characteristics of this new variety when grown under normal horticultural practices in Kamifurano, Hokkaido, Japan.

1. Cylindrical plant shape;

2. Large bracts; and

3. A fruity, tropical fruity flavor.

### DESCRIPTION OF THE PHOTOGRAPHS

This hop plant is illustrated by the accompanying photographs, which show the plant's overall plant shape, leaf shape and cone shape. The photographs are of three-year-old plants grown in Kamifurano, Hokkaido, Japan in August 2016. The colors shown are as true as can be reasonably obtained by conventional photographic procedures.

FIG. 1 shows the plant shape of ‘Furano K906901060 Go’.

FIG. 2 shows the leaf shape of ‘Furano K906901060 Go’ (left) and two commercial varieties in Japan. In the middle is ‘Little Star’, and on the right is ‘Furano Special’.

FIG. 3 shows the cone shape of ‘Furano K906901060 Go’.

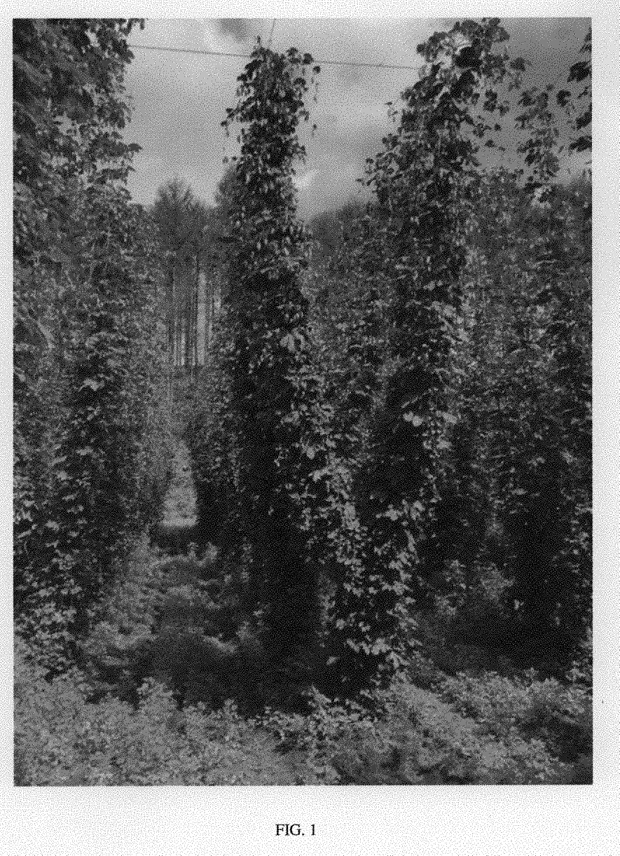

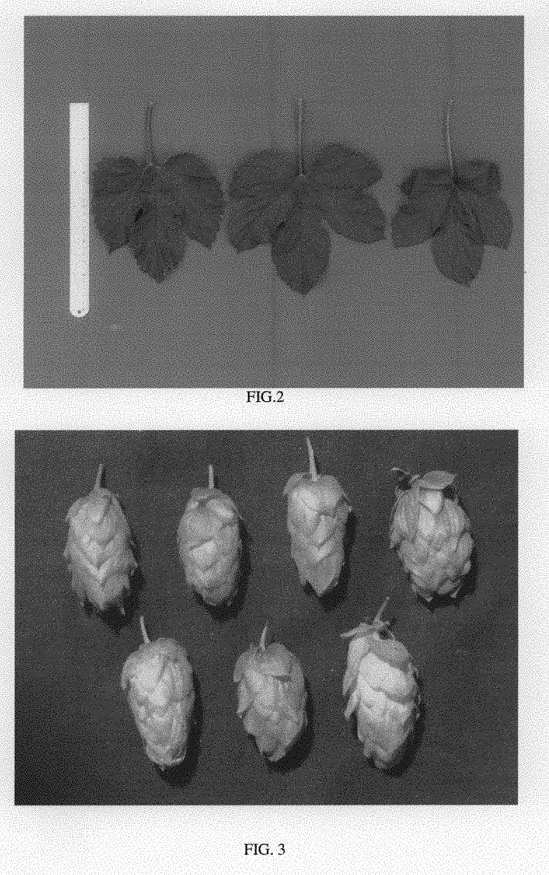

### DESCRIPTION OF THE NEW VARIETY

The following detailed descriptions set forth the distinctive characteristics of ‘Furano K906901060 Go’. The data, which define these characteristics, were collected from asexual reproductions carried out in Kamifurano, Hokkaido, Japan. Data was collected on mature plants in Kamifurano, Hokkaido, Japan. Color references are to Japan Horticultural Plant Color Chart (1987, Japan Color Research Institute).
Classification:
   Family.—Cannabaceae.
   Classification.—Humulus lupulus L.
   Common name.—Hop.
Propagation:
   Type.—Vegetative cuttings.
Plant description:
   Plant and growth habit.—The plant growth type is normal, not dwarf.
   Plant shape.—Cylindrical.
   Plant height.—5.5 m when grown on a trellis.
Lateral branch description:
   Length.—Average of 0.53 m.
   Internode length.—14.9 cm.
Foliage description:
   Length.—Average is 16.2 cm (2 year mean).
   Color, upper surface of fully expanded leaves.—Deep yellowish green (Japan Color Chart No. 3706).
   The number of lobes.—Three to five.
   Blistering.—Upper surface: Weak to medium.
Inflorescence buds:
   Cone size.—Medium.
   Cone length.—39.8 mm.
   Cone shape.—Ovate.
   Weight of one hundred cones.—Medium (21.3 g in dry matter).
   Color.—Bright yellowish green (Japan Color Chart No. 3704).
   Flowering season.—Early, around July 5^{th}.
Spike bracts:
   Length.—Large (20.3 mm).
   Width.—10.7 mm.
Pedicels: Not observed.
Reproductive organs:
   Stamens.—Not observed.
   Pistils.—Not observed.
Disease and pest/insect resistance: Field resistant against hop downey mildew. Not resistant to hop powdery mildew.
Brewing characteristics (2015): Content of alpha acid is 4.1% (moisture content is adjusted to 11%). Content of beta acid is 6.2% (moisture content is adjusted to 11%). Ratio content of beta acids to alpha acids is 1.5. Content of humulene is 22.2%. Content of caryophyllene is 20.6%. Ratio content of humulene to caryophyllene is 1.08. Content of myrcene is 51.8%. Content of farnesene is 0.3%. Content of linalool is 2.7%. Ratio of co-alpha to all alpha acids is 15%. Flavor of ‘Furano K906901060 Go’ is fruity, such as mango or tropical fruits.
   Storage stability.—75.4% after 6 months at room temperature.
Yield: 1.36 tons per hectare.

We claim:

1. A new and distinct variety of hop plant named ‘Furano K906901060 Go’ as illustrated and described herein.
